(19) United States
(12) Patent Application Publication
(10) Pub. No.: US 2007/0298443 A1
(43) Pub. Date: Dec. 27, 2007

(54) Screening Method Using Antibody Heavy Chains

(76) Inventor: Yiyou Chen, San Jose, CA (US)

Correspondence Address: Elena E. Quertermous;DANISCO US INC., GENENCOR DIVISION, 925 Page Mill Road, Palo Alto, CA, 94304, US

(21) Appl. No.: 10/594,190

(22) PCT Filed: May 12, 2005
(86) PCT No.: PCT/US05/16695

§ 371 Date: Jun. 27, 2007

Related U.S. Application Data
(60) Provisional application No. 60/577,303, filed on Jun. 4, 2004.

(51) Int. Cl. G01N 33/53; C12N 15/13; G01N 33/566

(52) U.S. Cl. 435/7.23; 435/69.3; 435/7.21

(57) ABSTRACT

The present invention relates to a method of screening which includes an antibody heavy chain and a reporter gene, such as a camel antibody and beta-lactamase, respectively.

### FIELD OF THE INVENTION

The present invention relates to a method of screening which includes an antibody heavy chain and a reporter gene, such as a camel antibody and beta-lactamase, respectively, and use of the method in diagnosis and therapy.

### BACKGROUND

Identification of tumor antigens is a time consuming and labor intensive process. Classical methods involve immunizing mice or other rodents with either tumor cells or tumor extracts. B cells from these mice are then fused with specific tumor cells to generate immortalized B cell hybridomas which secret monoclonal antibodies into the culture supernatant. Binding specificity of these antibodies can be confirmed by a combination of methods, including western blot, FACS and immunohistochemistry.

However, a serious drawback to this approach is the low efficiency in generating hybridomas, which often results in the loss of antigen specific antibodies, especially when complex antigens are used. Newer approaches have also been used to circumvent this problem by cloning the antibody genes via RT-PCR and expressing the recombinant antibody proteins in other host cells. However, the original pair configuration between the heavy chain and light chain can get scrambled during the cloning process. As a result, vastly more clones need to be screened to cover the original antibody repertoire (for example, >10,000 clones need to be screened in order to cover the diversity encoded by 100 different B cells).

Traditional approaches are often inconsistent and time consuming.

### SUMMARY OF THE INVENTION

In a first aspect, the invention is drawn to a method for identifying at least one antigen or antigen binder comprising:
i) immunizing a camelid;
ii) isolating at least one V_{H}H gene from the immunized camelid;
iii) fusing the at least one V_{H}H gene to a reporter gene, thereby creating at least one fusion gene;
iv) transforming the at least one fusion gene into a species that permits secretion of at least one fusion protein from the at least one fusion gene;
v) incubating the at least one fusion protein with at least one target and
vi) identifying the at least one antigen or antigen binder.

In a second aspect, the invention is drawn to at least one isolated antigen or antigen binder, the antigen or antigen binder isolated by a method comprising:
i) immunizing a camelid;
ii) isolating at least one V_{H}H gene from the immunized camelid;
iii) fusing the at least one V_{H}H gene to a reporter gene, thereby creating at least one fusion gene;
iv) transforming the at least one fusion gene into a species that permits secretion of at least one fusion protein from the at least one fusion gene;
v) incubating the at least one fusion protein with at least one target and
vi) identifying the at least one isolated antigen or antigen binder.

In a third aspect, the invention is drawn to a method of quantifying antigen amount on a target, comprising:
i) immunizing a camelid;
ii) isolating at least one V_{H}H gene from the immunized camelid;
iii) fusing the at least one V_{H}H gene to a reporter gene, thereby creating at least one fusion gene;
iv) transforming the at least one fusion gene into a species that permits secretion of at least one fusion protein from the at least one fusion gene;
v) incubating the at least one fusion protein with at least one target;
vi) measuring binding between the at least one target and the at least one fusion protein and
vii) quantifying antigen amount.
   In a preferred embodiment, step vii) further characterizes determining antigen density.

In a fourth aspect, the invention is drawn to a method of determining affinity, comprising:
i) immunizing a camelid;
ii) isolating at least one V_{H}H gene from the immunized camelid;
iii) fusing the at least one V_{H}H gene to a reporter gene, thereby creating at least one fusion gene;
iv) transforming the at least one fusion gene into a species that permits secretion of at least one fusion protein from the at least one fusion gene;
v) incubating the at least one fusion protein with at least one target;
vi) measuring affinity between the at least one target and the at least one fusion protein.

In preferred embodiments of the aspects, the camelid comprises either a camel or a llama. In a preferred embodiment, the camelid is a camel. In a preferred embodiment, the camelid is a llama. In a preferred embodiment, immunizing occurs with whole cells, cell membrane fractions and peptides specific to an antigen of interest, for example CEA, Muc-1, Tag72, αVβ3 and αVP5. In a preferred embodiment, immunizing occurs with tumour extracts.

In preferred embodiments of the aspects, the at least one V_{H}H gene is isolated with RT-PCR. In preferred embodiments of the aspects, the species is E. Coli. In preferred embodiments of the aspects, the target is at least one cancer cell line. (see, for a list of additional targets, WO 03/105757 and WO 03/107009, both of which are incorporated by reference, herein, including any drawings).

In preferred embodiments of the aspects, the at least one antigen or antigen binder is identified by measuring activity of the fusion protein. In preferred embodiments of the aspects, the reporter gene is a BLA. In preferred embodiments of the aspects, activity is determined with a nitrocefin assay as disclosed in the Examples.

In a preferred embodiments of the aspects, binding is measured with FACS, ELISA or IHC. In a preferred embodiment, binding is measured with FACS. In a preferred embodiment, binding is measured with ELISA. In a preferred embodiment, binding is measured with IHC.

### BRIEF DESCRIPTION OF THE FIGURES

FIG. 1 sets forth the amino acid sequence for the beta-lactamase protein.

FIG. 2 shows some typical antibody structure disclosing, for example the heavy and light chains. For additional description, especially as it relates to a VHH, (see U.S. Pat. Nos. 6,005,079 and 5,874,541, which is incorporated by reference herein, including any drawings).

FIG. 3 shows the plasmid map for pNA31.1 plasmid which will be used for creating Llama vHH expression library in E. coli. The vHH gene repertoire will be fused in-frame with upstream pelB signal sequence and downstream BLA sequence upon digestion of both vHH PCR fragments and vector pNA31.1 with NcoI and PinAI enzymes. The expression will be driven by lacP and terminated by T7 terminator, as shown.

FIG. 4 shows the complete nucleotide sequence of plasmid pNA31.1.

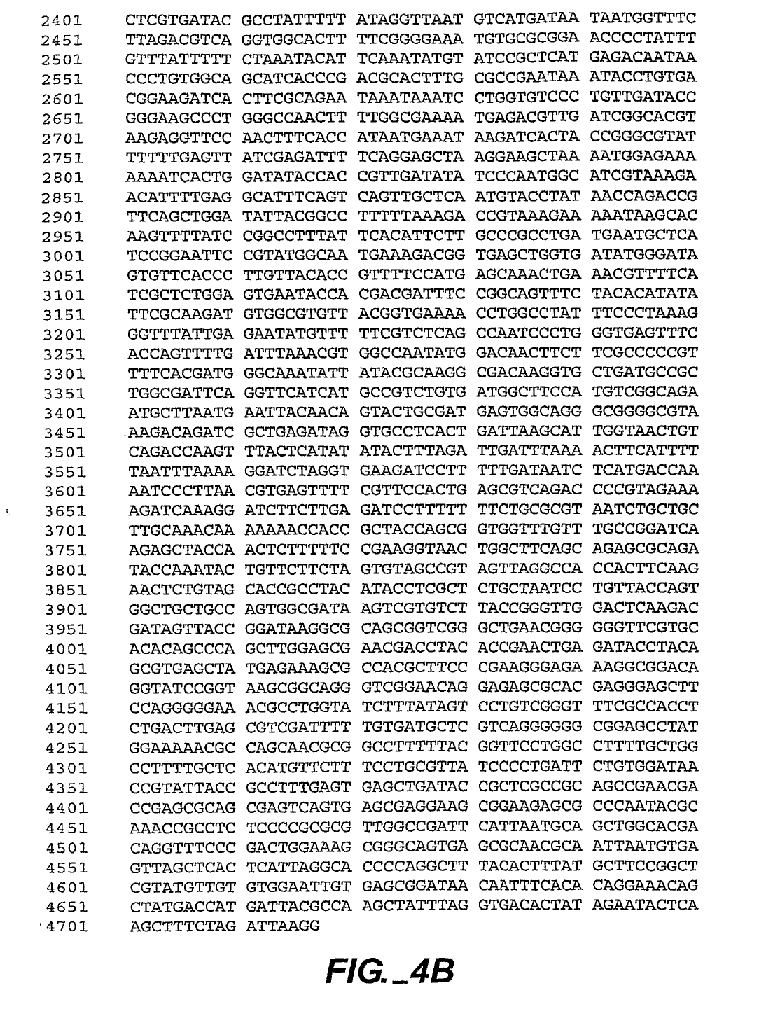

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the present invention, the following terms are used as described below.

The term “camelid” shall include, as examples, old world camelids (e.g., Camelus bactrianus and Camelus dromaderius) and new world camelids (e.g., Lama paccos, Lama glama and Lama vicugna). Examples of camlids within the scope of the invention include camels and llamas.

The term “reporter” shall refer to a portion of a molecule, such as a portion of a fusion protein, as disclosed in the invention, which allows quantification of a property, such as enzymatic activity, of the molecule. The non-limiting example of beta-lactamase (BLA) as a reporter is disclosed herein.

The term “reporter gene” is used herein to designate a gene that encodes a molecule that is a reporter.

The term “gene” as used herein is used to designate a molecule comprised of two or more deoxyribonucleotides or ribonucleotides. The exact size will depend on many factors, which in turn depends on the ultimate function or use of the oligonucleotide. Genes can be prepared by any suitable method, including, for example, cloning and restriction of appropriate sequences and direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Lett. 22:1859-1862; and the solid support method of U.S. Pat. No. 4,458,066, each incorporated herein by reference. A review of synthesis methods is provided in Goodchild, 1990, Bioconjugate Chemistry 1(3):165-187, incorporated herein by reference.

The term “fusion gene” is used herein to designate a gene construct that results when any one gene is fused to another. All known fusion methods are intended to be within the scope of the invention.

The term “protein” is used interchangeably here, as well as in the art, with the terms “peptide” and “polypeptide,” and refers to a molecule comprising two or more amino acid residues joined by a peptide bond.

Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine), nonpolar side chains (e.g., alanine, valine, leucine, isoleucine, proline, phenylalanine, methionine, tryptophan, cysteine, glycine), beta-branched side chains (e.g., threonine, valine, isoleucine) and aromatic side chains (e.g., tyrosine, phenylalanine, tryptophan, histidine). Standard three-letter or one-letter amino acid abbreviations may be used in this application, as well as in the art. One skilled in the art may make equivalent substitutions (e.g., an aromatic substituted for an aromatic) and such equivalent substations are intended to be within the scope of the claims, where appropriate.

The peptides, polypeptides and proteins of the invention can also comprise one or more non-classical amino acids. Non-classical amino acids include, but are not limited to, the D-isomers of the common amino acids, α-amino isobutyric acid, 4-aminobutyric acid (4-Abu), 2-aminobutyric acid (2-Abu), 6-amino hexanoic acid (Ahx), 2-amino isobutyric acid (2-Aib), 3-amino propionoic acid, omithine, norleucine, norvaline, hydroxyproline, sarcosine, citrulline, cysteic acid, t-butylglycine, t-butylalanine, phenylglycine, cyclohexylalanine, β-alanine, fluoro-amino acids and designer amino acids such as β-methyl amino acids, Cα-methyl amino acids and Nα-methyl amino acids.

The term “fusion protein” is used herein to designate a protein that results when any protein is fused to another. A fusion protein may also result when one gene is fused to another in an effort to create a fusion protein, and then the resulting fusion gene is expressed. All known fusion methods are intended to be within the scope of the invention.

The term “binder,” as used herein, shall refer to a molecule that has been determined to bind to a V_{H}H protein, as described herein. This could be verified by any known method which measures binding. All binding affinities are intended to be within the contemplated scope of the invention depending upon the purpose of the contemplated assay.

The terms “cell”, “cell line”, and “cell culture” can be used interchangeably and all such designations include progeny.

The terms “transformants” or “transformed cells” include the primary transformed cell and cultures derived from that cell without regard to the number of transfers. All progeny may not be precisely identical in DNA content due to deliberate or inadvertent mutations. Mutant progeny that have the same functionality as screened for in the originally transformed cell are included in the definition of transformants. The cells can be prokaryotic or eukaryotic.

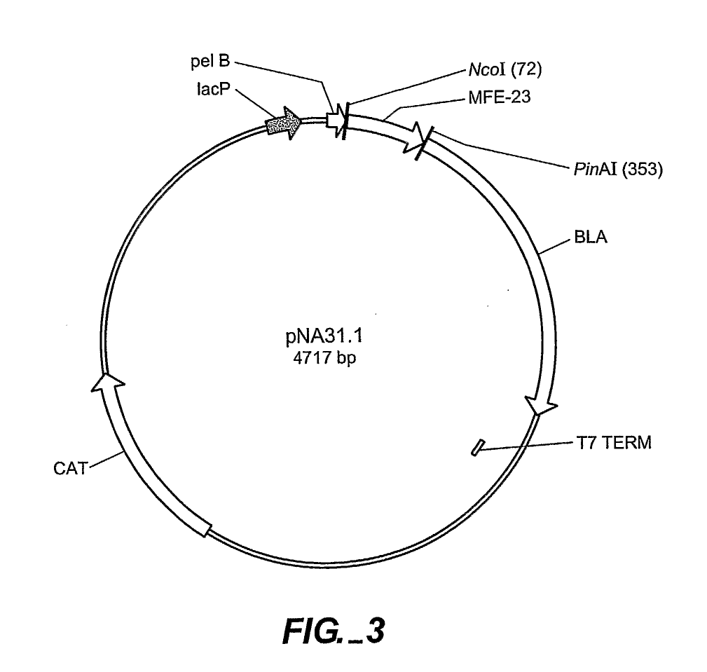

The term “Ab” or “antibody” refers to polyclonal and monoclonals antibodies, chimeric antibodies, humanized antibodies, human antibodies, immunoglobulins or antibody or functional fragments of an antibody that bind to an antigen. Examples of such functional entities include complete antibody molecules, antibody fragments, such as Fv, single chain Fv, complementarity determining regions (CDRs), V_{L} (light chain variable region), V_{H} (heavy chain variable region) and any combination of those or any other functional portion of an immunoglobulin peptide capable binding to target antigen. (see, for example, FIG. 2).

The term “V_{H}H” refers to the heavy chain antibody portion, specifically, for example, the heavy chain antibody portion of a camelid. (see, e.g., U.S. Pat. Nos. 6,005,079 and 5,874,541, both of which are incorporated by reference herein, including any drawings).

The term “target” refers to a substance of interest against which a fusion protein, as disclosed herein, may be incubated so that an antigen binder or antigen of interest may be identified according to the methods of the invention. As non-limiting examples, a target may include a cancerous cell, cell line or cell culture, tumour extracts or a cancerous tissue or organ, a molecule associated with a cancerous cell, cell line or cell culture, tumour extracts or a cancerous tissue or organ, or a cell, cell line or cell culture, tissue or organ associated with a cancerous cell, cell line or cell culture, tumour extracts or a cancerous tissue or organ.

The term “tumour extract” shall refer to an isolate from a cancerous cell, cell line or cell culture or a cancerous tissue or organ.

The term “antigen” refers to a molecule that binds an antibody, as defined herein. As an example, an antigen of interest, according to the invention, may be a cancer antigen whose overexpression is correlated with a specific pathology, such as, for example, a specific indication of cancer.

In a first aspect, the invention is drawn to a method for identifying at least one antigen or antigen binder comprising:
i) immunizing a camelid;
ii) isolating at least one V_{H}H gene from the immunized camelid;
iii) fusing the at least one V_{H}H gene to a reporter gene, thereby creating at least one fusion gene;
iv) transforming the at least one fusion gene into a species that permits secretion of at least one fusion protein from the at least one fusion gene;
v) incubating the at least one fusion protein with at least one target and
vi) identifying the at least one antigen or antigen binder.

In a second aspect, the invention is drawn to at least one isolated antigen or antigen binder, the antigen or antigen binder isolated by a method comprising:
i) immunizing a camelid;
ii) isolating at least one V_{H}H gene from the immunized camelid;
iii) fusing the at least one V_{H}H gene to a reporter gene, thereby creating at least one fusion gene;
iv) transforming the at least one fusion gene into a species that permits secretion of at least one fusion protein from the at least one fusion gene;
v) incubating the at least one fusion protein with at least one target and
vi) identifying the at least one isolated antigen or antigen binder.

In a third aspect, the invention is drawn to a method of quantifying antigen amount on a target, comprising:
i) immunizing a camelid;
ii) isolating at least one V_{H}H gene from the immunized camelid;
iii) fusing the at least one V_{H}H gene to a reporter gene, thereby creating at least one fusion gene;
iv) transforming the at least one fusion gene into a species that permits secretion of at least one fusion protein from the at least one fusion gene;
v) incubating the at least one fusion protein with at least one target;
vi) measuring binding between the at least one target and the at least one fusion protein and
vii) quantifying antigen amount.
   In a preferred embodiment, step vii) further characterizes determining antigen density.
In a fourth aspect, the invention is drawn to a method of determining affinity, comprising:
i) immunizing a camelid;
ii) isolating at least one V_{H}H gene from the immunized camelid;
iii) fusing the at least one V_{H}H gene to a reporter gene, thereby creating at least one fusion gene;
iv) transforming the at least one fusion gene into a species that permits secretion of at least one fusion protein from the at least one fusion gene;
v) incubating the at least one fusion protein with at least one target;
vi) measuring affinity between the at least one target and the at least one fusion protein.

In preferred embodiments of the aspects, the camelid comprises either a camel or a llama. In a preferred embodiment, the camelid is a camel. In a preferred embodiment, the camelid is a llama. In a preferred embodiment, immunizing occurs with whole cells, cell membrane fractions and peptides specific to an antigen of interest, for example CEA, Muc-1, Tag72, αVβ3 and αVβ5. In a preferred embodiment, immunizing occurs with tumor extracts.

Camel V_{H}H antibodies are composed of only a heavy chain and lack light chains (see, e.g., FIG. 2; also see, e.g., U.S. Pat. Nos. 6,005,079 and 5,874,541, both of which are incorporated by reference herein, including any drawings). As a result, it is easier to cover the entire antibody repertoire (for the above example, only 100 clones are needed to cover 100 V_{H}H encoding B cells). Also, a V_{H}H-reporter (e.g., BLA) fusion construct virtually eliminates background in the cloning step. Further, unlike other affinity tags that require a secondary reagent for detection, BLA provides a convenient way to directly monitor antibody binding as enzymatic activity of a V_{H}H-BLA will correlate linearly with the amount of V_{H}H binding, which can be used to determine antigen density on target cells or cell extracts. Likewise, the 1:1 relationship between V_{H}H and BLA allows fairly accurate determination of antibody off-rate that provides information of antibody affinity.

In preferred embodiments of the aspects, the at least one V_{H}H gene is isolated with RT-PCR.

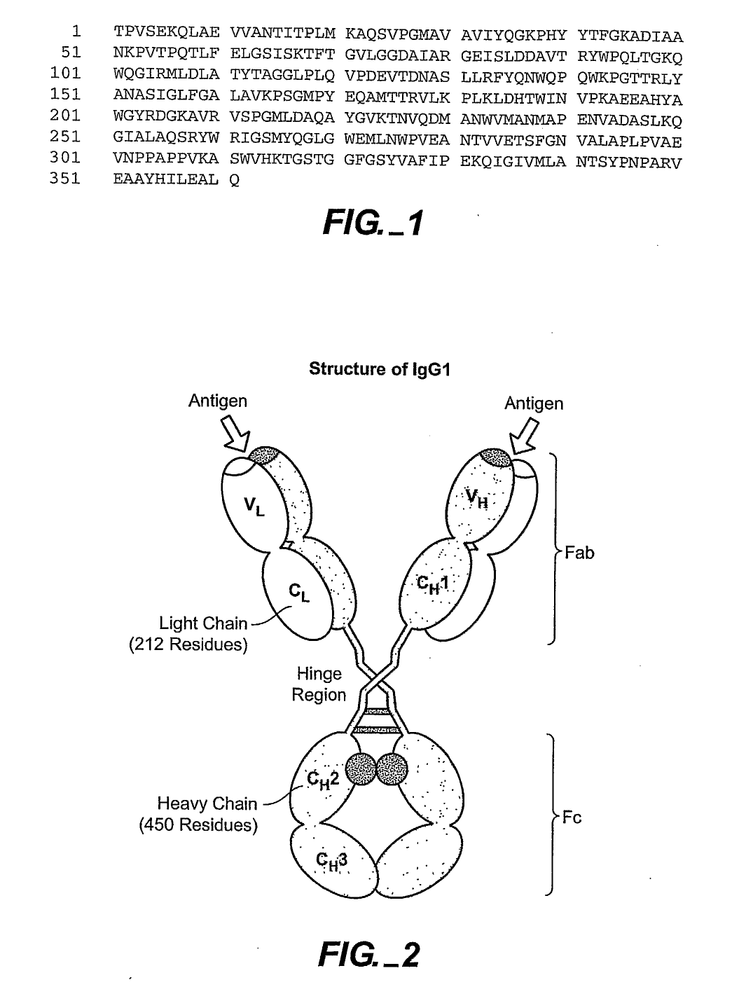

In preferred embodiments of the aspects, the reporter gene is a BLA. A representative example of a BLA sequence is depicted in FIG. 1.

BLA enzymes are widely distributed in both gram-negative and gram-positive bacteria. BLA enzymes vary in specificity, but have in common that they hydrolyze β-lactams, producing substituted β-amino acids. Thus, they confer resistance to antibiotics containing β-lactams. Because BLA enzymes are not endogenous to mammals, they are only minimally subject to interference from inhibitors, enzyme substrates or endogenous enzyme systems (e.g., unlike proteases) and therefore are particularly well suited for reporter function.

Examples of specific BLAs contemplated according to the current invention include, but are not limited to, Class A, B, C or D β-lactamase, β-galactosidase, see Benito et al., FEMS Microbiol. Lett. 123:107 (1994), fibronectin, glucose oxidase, glutathione S-transferase, see Napolitano et al., Chem. Biol. 3:359 (1996) and tissue plasminogen activator, see Smith et al., J. Biol. Chem. 270:30486 (1995).

In one embodiment of the invention, the reporter gene comprises an alkaline phosphatase that converts a 4′-phosphate derivative of the epipodophyl-lotoxin glucosides into an active drug. Such derivatives include etoposide-4′-phosphate, etoposide-4′-thiophosphate and teniposide-4′-phosphate. Other embodiments of the invention may include phosphate derivatives of these glucosides wherein the phosphate moiety is placed at other hydroxyl groups on the glucosides.

In preferred embodiments of the aspects, the species is E. coli. Microbial strains other than E. coli can also be used, such as bacilli, for example Bacillus subtilis, various species of Pseudomonas and Salmonella, and other bacterial strains. In such procaryotic systems, plasmid vectors that contain replication sites and control sequences derived from the host or a species compatible with the host are typically used.

For expression of constructions under control of most bacterial promoters, E. coli K12 strain MM294, obtained from the E. coli Genetic Stock Center under GCSC #6135, can be used as the host. For expression vectors with the P_{L}N_{RBS} or P_{L} T7_{RBS} control sequence, E. coli K12 strain MC1000 lambda lysogen, N₇N₅₃cI857 SusP₈₀, ATCC 39531, may be used. E. coli DG116, which was deposited with the ATCC (ATCC 53606) on Apr. 7, 1987, and E. coli KB2, which was deposited with the ATCC (ATCC 53075) on Mar. 29, 1985, are also useful host cells. For M13 phage recombinants, E. coli strains susceptible to phage infection, such as E. coli K12 strain DG98 (ATCC 39768), are employed. The DG98 strain was deposited with the ATCC on Jul. 13, 1984.

E. coli may be typically transformed, for example, using derivatives of pBR322, described by Bolivar et al., 1977, Gene 2:95. Plasmid pBR322 contains genes for ampicillin and tetracycline resistance. These drug resistance markers can be either retained or destroyed in constructing the desired vector and so help to detect the presence of a desired recombinant. Commonly used procaryotic control sequences, i.e., a promoter for transcription initiation, optionally with an operator, along with a ribosome binding site sequence, include the β-lactamase (penicillinase) and lactose (lac) promoter systems, see Chang et al., 1977, Nature 198:1056, the tryptophan (trp) promoter system, see Goeddel et al., 1980, Nuc. Acids Res. 8:4057, and the lambda-derived P_{L} promoter, see Shimatake et al., 1981, Nature 292:128, and gene N ribosome binding site (N_{RBS}). A portable control system cassette is set forth in U.S. Pat. No. 4,711,845, issued Dec. 8, 1987. This cassette comprises a P_{L} promoter operably linked to the N_{RBS} in turn positioned upstream of a third DNA sequence having at least one restriction site that permits cleavage within six base pairs 3′ of the N_{RBS} sequence. Also useful is the phosphatase A (phoA) system described by Chang et al., in European Patent Publication No. 196,864, published Oct. 8, 1986. However, any available promoter system compatible with procaryotes can be used to construct a expression vector of the invention.

In addition to bacteria, eucaryotic microbes, such as yeast, can also be used as the species. Laboratory strains of Saccharonzyces cerevisiae, Baker's yeast, are most often used, although a number of other strains are commonly available. While vectors employing the two micron origin of replication are common, see Broach, 1983, Meth. Enz. 101:307, other plasmid vectors suitable for yeast expression are known. See, e.g., Stinchcomb et al., 1979, Nature 282:39; Tschempe et al., 1980, Gene 10:157; and Clarke et al., 1983, Meth. Enz. 101:300. Control sequences for yeast vectors include promoters for the synthesis of glycolytic enzymes. See Hess et al., 1968, J. Adv. Enzyme Reg. 7:149; Holland et al., 1978, Biotechnology 17:4900; and Holland et al., 1981, J. Biol. Chem. 256:1385. Additional promoters known in the art include the promoter for 3-phosphoglycerate kinase, see Hitzeman et al., 1980, J. Biol. Chem. 255:2073, and those for other glycolytic enzymes, such as glyceraldehyde 3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase and glucokinase. Other promoters that have the additional advantage of transcription controlled by growth conditions are the promoter regions for alcohol dehydrogenase 2, isocytochrome C, acid phosphatase, degradative enzymes associated with nitrogen metabolism and enzymes responsible for maltose and galactose utilization.

Terminator sequences may also be used to enhance expression when placed at the 3′ end of the coding sequence. Such terminators are found in the 3′ untranslated region following the coding sequences in yeast-derived genes. Any vector containing a yeast-compatible promoter, origin of replication and other control sequences is suitable for use in constructing yeast expression vectors.

The coding sequence can also be expressed in eucaryotic host cell cultures derived from multicellular organisms. See, e.g., Tissue Culture, Academic Press, Cruz and Patterson, editors (1973). Useful host cell lines include COS-7, COS-A2, CV-1, murine cells such as murine myelomas N51 and VERO, HeLa cells and Chinese hamster ovary (CHO) cells. Expression vectors for such cells ordinarily include promoters and control sequences compatible with mammalian cells such as, for example, the commonly used early and late promoters from Simian Virus 40 (SV 40), see Fiers et al., 1978, Nature 273:113 or other viral promoters such as those derived from polyoma, adenovirus 2, bovine papilloma virus (BPV) or avian sarcoma viruses or immunoglobulin promoters and heat shock promoters.

Enhancer regions are also important in optimizing expression. Origins of replication may be obtained, if needed, from viral sources.

The species may also include plant cells, and control sequences compatible with plant cells, such as the nopaline synthase promoter and polyadenylation signal sequences, see Depicker et al., 1982, J. Mol. Appl. Gen. 1:561, are available. Expression systems employing insect cells utilizing the control systems provided by baculovirus vectors have also been described. See Miller et al., in Genetic Engineering (1986), Setlow et al., eds., Plenum Publishing, Vol. 8, pp. 277-97. Insect cell-based expression can be accomplished in Spodoptera frugipeida. These systems are also successful in producing recombinant enzymes.

Depending on the species, transformation is done using standard techniques appropriate to such cells. The calcium treatment employing calcium chloride, as described by Cohen, 1972, Proc. Natl. Acad. Sci. USA 69:2110, is used for procaryotes or other cells that contain substantial cell wall barriers. Infection with Agrobacterium tumefaciens, see Shaw et al., 1983, Gene 23:315, is used for certain plant cells. For mammalian cells, the calcium phosphate precipitation method of Graham et al, 1978, Virology 52:546 is preferred. Transformations into yeast are carried out according to the method of Van Solingen et al., 1977, J. Bact. 130:946, and Hsiao et al., 1979, Proc. Natl. Acad. Sci. USA 76:3829.

It may be desirable to modify the sequence of a DNA encoding a polypeptide to provide, for example, a sequence more compatible with the codon usage of the species without modifying the amino acid sequence of the encoded protein. Such modifications to the initial 5-6 codons may improve expression efficiency. DNA sequences which have been modified to improve expression efficiency, but which encode the same amino acid sequence, are considered to be equivalent and encompassed by the present invention.

A variety of site-specific primer-directed mutagenesis methods are available and well-known in the art. See, e.g., Sambrook et al., Molecular Cloning: A Laboratory Manual, Cold Spring Harbor, 1989, second edition, chapter 15.51, “Oligonucleotide-mediated mutagenesis,” which is incorporated herein by reference. The polymerase chain reaction (PCR) can be used to perform site-specific mutagenesis. In another technique now standard in the art, a synthetic oligonucleotide encoding the desired mutation is used as a primer to direct synthesis of a complementary nucleic acid sequence contained in a single-stranded vector, such as pBSM13+ derivatives, that serves as a template for construction of the extension product of the mutagenizing primer. The mutagenized DNA is transformed into a host bacterium, and cultures of the transformed bacteria are plated and identified. The identification of modified vectors may involve transfer of the DNA of selected transformants to a nitrocellulose filter or other membrane and the “lifts” hybridized with kinased synthetic mutagenic primer at a temperature that permits hybridization of an exact match to the modified sequence but prevents hybridization with the original unmutagenized strand. Transformants that contain DNA that hybridizes with the probe are then cultured (the sequence of the DNA is generally confirmed by sequence analysis) and serve as a reservoir of the modified DNA.

Because of the redundancy in the genetic code, typically a large number of DNA sequences encode any given amino acid sequence and are, in this sense, equivalent. As described below, it may be desirable to select one or another equivalent DNA sequences for use in a expression vector, based on the preferred codon usage of the host cell into which the expression vector will be inserted. The present invention is intended to encompass all DNA sequences that encode disclosed proteins.

An operable expression clone may be used and is constructed by placing the coding sequence in operable linkage with a suitable control sequence in an expression vector. The vector can be designed to replicate autonomously in the host cell or to integrate into the chromosomal DNA of the host cell. The resulting clone is used to transform a suitable host, and the transformed host is cultured under conditions suitable for expression of the coding sequence.

Construction of suitable clones containing the coding sequence and a suitable control sequence employ standard ligation and restriction techniques that are well understood in the art. In general, isolated plasmids, DNA sequences or synthesized oligonucleotides are cleaved, modified and religated in the form desired. Suitable restriction sites can, if not normally available, be added to the ends of the coding sequence so as to facilitate construction of an expression clone.

Site-specific DNA cleavage is performed by treating with a suitable restriction enzyme (or enzymes) under conditions that are generally understood in the art and specified by the manufacturers of commercially available restriction enzymes. See, e.g., product catalogs from Amersham (Arlington Heights, Ill.), Roche Molecular Biochemicals (Indianapolis, Ind.), and New England Biolabs (Beverly, Mass.). Incubation times of about one to two hours at a temperature that is optimal for the particular enzyme are typical. After each incubation, protein is removed by extraction with phenol and chloroform; this extraction can be followed by ether extraction and recovery of the DNA from aqueous fractions by precipitation with ethanol. If desired, size separation of the cleaved fragments may be performed by polyacrylamide gel or agarose gel electrophoresis using standard techniques. See, e.g., Maxam et al., 1980, Methods in Enzymology 65:499-560.

Ligations can be performed, for example, in 15-30 μl volumes under the following standard conditions and temperatures: 20 mM Tris-Cl, pH 7.5, 10 mM MgCl₂, 10 mM DTT, 33 μg/ml BSA, 10-50 mM NaCl, and either 40 μM ATP and 0.01-0.02 (Weiss) units T4 DNA ligase at 0° C. (for ligation of fragments with complementary single-stranded ends) or 1 mM ATP and 0.3-0.6 units T4 DNA ligase at 14° C. (for “blunt end” ligation). Intermolecular ligations of fragments with complementary ends are usually performed at 33-100 μg/ml total DNA concentrations (5-100 nM total ends concentration). Intermolecular blunt end ligations (usually employing a 20-30 fold molar excess of linkers, optionally) are performed at 1 μM total ends concentration.

Correct ligations for plasmid construction can be confirmed using any suitable method known in the art. For example, correct ligations for plasmid construction can be confirmed by first transforming a suitable host, such as E. coli strain DG101 (ATCC 47043) or E. coli strain DG116 (ATCC 53606), with the ligation mixture. Successful transformants are selected by ampicillin, tetracycline or other antibiotic resistance or sensitivity or by using other markers, depending on the mode of plasmid construction, as is understood in the art. Plasmids from the transformants are then prepared according to the method of Clewell et al., 1969, Proc. Natl. Acad. Sci. USA 62:1159, optionally following chloramphenicol amplification. See Clewell, 1972, J. Bacteriol. 110:667. Alternatively, plasmid DNA can be prepared using the “Base-Acid” extraction method at page 11 of the Bethesda Research Laboratories publication Focus 5 (2), and very pure plasmid DNA can be obtained by replacing steps 12 through 17 of the protocol with CsCl/ethidium bromide ultracentrifugation of the DNA. As another alternative, a commercially available plasmid DNA isolation kit, e.g., HISPEED™, QIAFILTER™ and QIAGEN® plasmid DNA isolation kits (Qiagen, Valencia Calif.) can be employed following the protocols supplied by the vendor. The isolated DNA can be analyzed by, for example, restriction enzyme digestion and/or sequenced by the dideoxy method of Sanger et al., 1977, Proc. Natl. Acad. Sci. USA 74:5463, as further described by Messing et al., 1981, Nuc. Acids Res. 9:309, or by the method of Maxam et al., 1980, Methods in Enzymology 65:499.

In a preferred embodiments of the aspects, activity is determined with a nitrocefin assay (as disclosed in the Examples and also see, for example, WO 03/105757 and WO 03/107009, both of which are incorporated by reference, herein, including any drawings).

In preferred embodiments of the aspects, the target is at least one cancer cell line. In another embodiment, the target is a cancer-related target that expresses CEA or that has CEA bound to itself or that has CEA located in its vicinity. In another preferred embodiment, the target is Muc-1 and Tag72 αVβ5. (see, for other targets, WO 03/105757 and WO 03/107009, both of which are incorporated by reference, herein, including any drawings).

Sources of cells or tissues include human, all other animals, bacteria, fungi, viruses and plant. Tissues are complex targets and refer to a single cell type, a collection of cell types or an aggregate of cells generally of a particular kind. Tissues may be intact or modified. General classes of tissue in humans include but are not limited to epithelial tissue, connective tissue, nerve tissue and muscle tissue.

In a preferred embodiments of the aspects, binding specificity is confirmed with FACS, ELISA or IHC. In a preferred embodiment, binding specificity is confirmed with FACS.

In a preferred embodiment, binding specificity is confirmed with ELISA. (see, for example, Yasuhito Abe, Teiri Sagawa, Ken Sakai and Shigeru Kimura. Enzyme-linked immunosorbent assay (ELISA) for human epidermal growth factor (hEGF). Clinica Chimica Acta, 168: 87-95, 1987; Yasuhito Abe, Masazumi Miyake, Teiri Sagawa and Shigeru Kimura. Enzyme-linked immunosorbent assay (ELISA) for human tumor 30 necrosis factor (hTNF). Clinica Chimica Acta 176: 213-218, 1988 amd Yasuhito Abe, Masazumi Miyake, Atsushi Horiuchi, Teiri Sagawa, Hitoshi Ono and Shigeru Kimura. Non-specific reaction in the sandwich immunoassay for human tumor necrosis factor-a (hTNF-a) Clinica Chimica Acta 181: 223-230, 1989, each of which is incorporated by reference herein.)

In a preferred embodiment, binding specificity is confirmed with IHC. (see, for example, Diagnostic Immunohistochemistry. David J. Dabbs. W.B Saunders Company. Philadelphia, Pa. 2001, which is incorporated by reference herein).

### EXAMPLES

### Example 1

### Immunization of Llama

Llamas may be immunized with whole cells, cell membrane fractions and peptides specific to an antigen of interest, for example CEA, Muc-1, Tag72, αVβ3 or αVβ5. Current methods are known for immunization with whole cells (see Current Protocols in Immunology (1995). John Wiley & Sonc, Inc. Pages: 2.5.1-2.5.17.).

Membrane fractions may be prepared by standard techniques. Cells may be homogenized or cavitated using the nitrogen bomb. Cell fractions may be separated using sequential centrifugations (Selection of ScFv phages on intact cells under low pH conditions leads to a significant loss of insert free phages. (2001). Tur M. K., Huhn S., Sasse S., Engert A. and Barth S. Biotechniques 30: 404-413).

Immunization with antigens may also be done with standard techniques.

Immunization may be done with 250 ug antigen in a water-in-oil emulsion using methods approved by the Animal Experimental Committee (Boersma W. J. A., Bogarts E. J. C., Bianch A. T. J., Claassen E. (1992) Adjuvant properties of stable water-in-oil emulsions: evaluation of the experience with specol. Res Immunol. 143:503.).

For example, llamas may be immunized with target cell lines ZR75-1 and T47D or 1918. The cell lines express the Muc1 and Tag72 antigens. A first immunization may be done using whole cells. Subsequent boosts may be done using membrane fractions to enrich the antibody repertoire to the cell surface antigens of interest. Immunization may occur in young adult llamas at 0, 21 and 35 days (Induction of immune responses and molecular cloning of the heavy chain antibody repertoire of Lama glama. (2000) van der Linden R, de Geus B, Stok W, Bos W, van Wassenaar D, Verrips T, Frenken L. J Immunol Methods. 240(1-2): 185-95).

As another example, llamas may be immunized with commercially available protein preparations of integrin CEA, Muc-1, Tag72, αVβ3 or αVβ5 following the immunization protocols specified above.

### Example 2

### Collection of Blood Samples from Llama

Peripheral blood samples are typically drawn from camelids from the jugular vein. The point of needle entry should be about half way between the dorsal and neck margin. This point avoids the thinner musculature and muchal ligament above it. Animals are restrained using a one-legged hobble and their heads are kept still to avoid injury to the operator. A syringe or evacuated collection tube can be used. The recommended needle is an 18 g×37 mm. Serum samples are processed as for any other mammal.

When serial samples are required the placing of an indwelling catheter may be the most convenient method. The catheter may be connected to an extension tube. The apparatus may be left filled with heparin:water, 1:10, held in place by simple sutures or “stitched” to the skin by drops of super glue.

### Example 3

### cDNA Preparation and PCR Amplification of Heavy Chain Fragments

RNA may be isolated from blood and lymph nodes according to the method described in Chomzeynski and Sachi, 1987. cDNA may be prepared on 100 μg total RNA with M-MLV Reverse Transcriptase (Gibco BRL) and hexanucleotide random primer (Amersham Biosciences) or oligo-dT primer as described before (de Haard et al., 1999). The cDNA may be purified with a phenol/chloroform extraction, combined with an ethanol precipitation and subsequently may be used as a template to specifically amplify the VHH repertoire. The complete heavy chain derived IgG genes from the Cameloid heavy chain antibodies (1.3-kB) and the conventional antibodies (1.65-kB) may be amplified with oligo-dT primer combined with FR1-specific primer HR-NBF1 (5′-GAGGTBCARCCATGGGASTCYGG-3′; bold indicates a NcoI site) on oligo-dT primed cDNA as template according to the methods described in EP01205100.9, which is herein incorporated by reference including any drawings. The heavy chain antibody derived IgG amplicon may be gel purified and used for cloning after digestion with NcoI enzyme introduced in HR-NBF1 primer and PinAI enzyme that may naturally occur in the FR4 region.

Alternatively, the vHH repertoire could be amplified in a hinge-dependent approach using two IgG-specific oligonucleotide primers as described in WO03050531A2. In a single PCR reaction, HR-NBF1 (5′-GAGGTBCARCCATGGGASTCYGG-3′; bold indicates a NcoI site) primer will be combined with a short HR-NBR1 (5′-AACAGTTAAGCTTCCGCTTACCGGTGGAGCTGGGGTCTTCGCTGTGGTGCG-3′; bold indicates a PinAI site) or long HR-NBR2 (5′-AACAGTTAAGCTTCCGCTTACCGGTTGGTTGTGGTTTTGGTGTCTTGGGTT-3′; bold indicates a PinAI site) hinge primer known to be specific for the amplification of heavy-chain variable region gene segments.

Please also see WO 03/050531A2, which is herein incorporated by reference, including any drawings. Please also refer to Reviews in Molecular Biotechnology 74(2001) 277-302 article by Serge Muyldermans for schematic overview of strategies to clone and select vHH genes from an immunized Ilama.

### Example 4

### Creation of vHH-BLA Expression Library

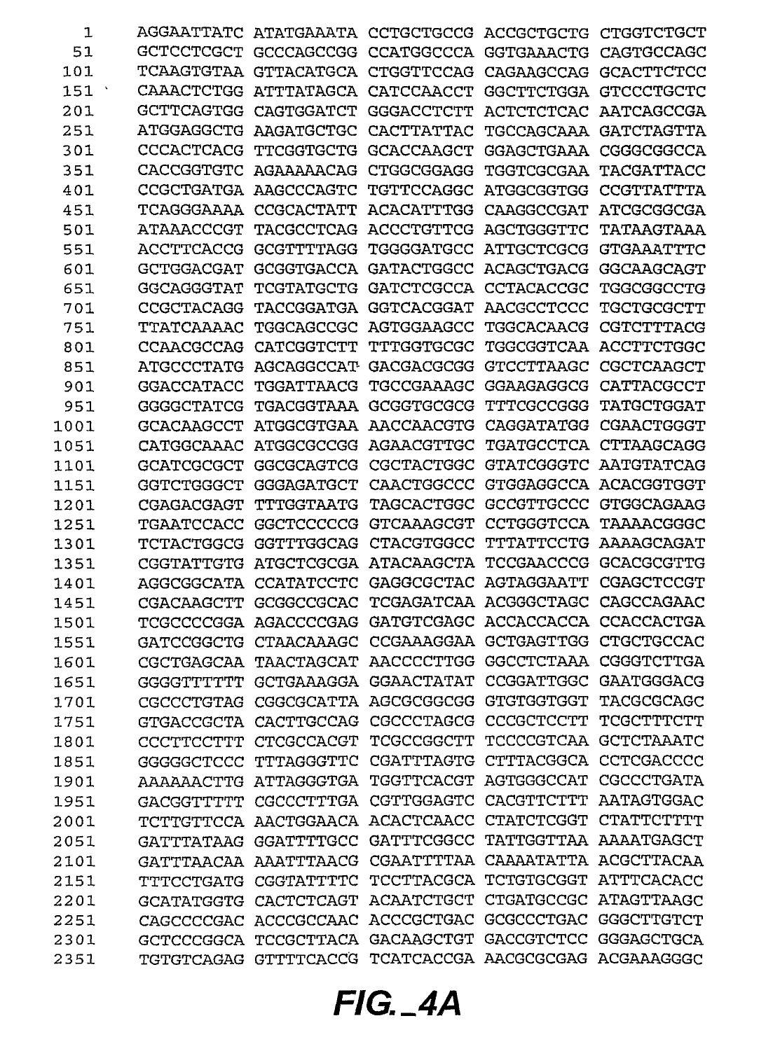

PCR-amplified vHH fragments of llama antibodies as described in example 3 above may be cloned into E. coli expression vector pNA31.1 as shown in FIG. 3. Plasmid pNA31.1 is a stuffer vector with an inactive BLA gene that was derived from plasmid pME27.1 (see, for example, CAB1, WO 03/105757 and WO 03/107009, both of which are incorporated by reference, herein, including any drawings) upon digestion with PstI enzyme to remove the 461-bp region containing a large part of MFE-23 scFv. Upon digestion of vHH PCR products obtained as described in Example 3 above and plasmid pNA31.1 with NcoI and PinAI enzymes, a 0.6-kb insert fragments and a 4.4-kb vector fragment, respectively, will be gel purified. They will then be ligated, followed by transformation into E. coli TOP10F′ (Invitrogen, Carlsbad, Calif.) competent cells and selection on LA+Cm10+0.1 CTX plates. Expression of vHH fragments as vHH-BLA fusion proteins will be driven by the lactose promoter (lacP), and the vHH-BLA fusion proteins will be targeted to the E. coli periplasm for secretion by the pelB signal sequence.

The heavy chain and the BLA domains may be fused together with a short linker sequence such as GGGGS or (GGGGS)2 in between them. Please refer to chapter 7 entitled ‘Single-chain Fv design and production by preparative folding’ by J. S. Huston et. al. in ‘Antibody Engineering’ book edited by Carl A. Borrebaeck (Second edition, Oxford University Press, 1995) for a discussion on various linkers successfully used in antibody engineering.

Transformants from TOP10F′ cells may be picked and inoculated in LB+10 ppm cmp in 96 well plates. They may be incubated at 30° C. for 48 hours. Bper reagent (PIERCE) may be added into each well and incubated at room temperature for 30 mins. Bper extract may be diluted in PBS and BLA activity will be measured using fluorogenic substrate nitrocefin (Oxoid).

### Example 5

### Incubation of Fusion Protein with Cancer Cells and Identification of Binding Clones

Cancer cells may be inoculated in 96 well plates and incubated for 24-48 hours at 37° C. They may be fixed by traditional formaldehyde fixation or ethanol fixation. Different concentration of Bper extracted fusion protein from Example 4 may be added into 96 well plates with cancer cells. The plate may be incubated at room temperature for 1 hour. Then unbound fusion protein may be washed away with PBST (PBS+0.1% Tween 20). Bound BLA may be measured by adding nitrocefin substrate into the 96 well plates. The clones that have the highest binding can be selected. A negative control of BLA can be included in the binding experiment so that a background of non-specific binding can be measured.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations that is not specifically disclosed. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the disclosed concepts may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

1. A method for identifying at least one antigen or antigen binder comprising:
i) immunizing a camelid;
ii) isolating at least one V_{H}H gene from the immunized camelid;
iii) fusing the at least one V_{H}H gene to a reporter gene, thereby creating at least one fusion gene;
iv) transforming the at least one fusion gene into a species that permits secretion of at least one fusion protein from the at least one fusion gene;
v) incubating the at least one fusion protein with at least one target and
vi) identifying the at least one antigen or antigen binder.

2. The method according to claim 1, wherein the camelid comprises either a camel or a llama.

3. The method according to claim 1, wherein immunizing occurs with whole cells, cell membrane fractions or peptides specific to an antigen of interest.

4. The method according to claim 3, wherein the antigen of interest comprises CEA, Muc-1, Tag72, αVβ3 or αVβ5.

5. The method according to claim 1, wherein immunizing occurs with tumour extracts.

6. The method according to claim 1, wherein the at least one V_{H}H gene is isolated with RT-PCR.

7. The method according to claim 1, wherein the species is E. Coli.

8. The method according to claim 1, wherein the at least one antigen or antigen binder is identified by measuring activity of the fusion protein.

9. The method according to claim 8, wherein the reporter gene is a BLA.

10. The method according to claim 9, wherein activity is determined with a nitrocefin assay.

11. The method according to claim 10, wherein binding is measured with FACS, ELISA or IHC.

12. The method according to claim 11, wherein binding is measured with FACS.

13. An antigen or antigen binder, the antigen or antigen binder isolated by a method comprising:
i) immunizing a camelid;
ii) isolating at least one V_{H}H gene from the immunized camelid;
iii) fusing the at least one V_{H}H gene to a reporter gene, thereby creating at least one fusion gene;
iv) transforming the at least one fusion gene into a species that permits secretion of at least one fusion protein from the at least one fusion gene;
v) incubating the at least one fusion protein with at least one target and
vi) identifying the at least one isolated antigen or antigen binder.

14. The antigen or antigen binder according to claim 13, wherein the antigen is CEA, Muc-1, Tag72, αVβ3 or αVβ5.

15. The method according to claim 13, wherein immunizing occurs with tumour extracts.

16. The method according to claim 13, wherein the at least one V_{H}H gene is isolated with RT-PCR.

17. The method according to claim 13, wherein the at least one antigen or antigen binder is identified by measuring activity of the fusion protein.

18. The method according to claim 17, wherein the reporter gene is a BLA.

19. The method according to claim 18, wherein activity is determined with a nitrocefin assay.

20. The method according to claim 13, wherein binding is measured with FACS, ELISA or IHC.

21. The method according to claim 20, wherein binding is measured with FACS.

22. A method of quantifying antigen amount on a target, comprising:
i) immunizing a camelid;
ii) isolating at least one V_{H}H gene from the immunized camelid;
iii) fusing the at least one V_{H}H gene to a reporter gene, thereby creating at least one fusion gene;
iv) transforming the at least one fusion gene into a species that permits secretion of at least one fusion protein from the at least one fusion gene;
v) incubating the at least one fusion protein with at least one target;
vi) measuring binding between the at least one target and the at least one fusion protein and
vii) quantifying antigen amount.

23. The method according to claim 22, wherein immunizing occurs with whole cells, cell membrane fractions and peptides specific to an antigen of interest.

24. The method according to claim 22, wherein the at least one antigen or antigen binder is identified by measuring activity of the fusion protein.

25. The method according to claim 22, wherein the reporter gene is a BLA.

26. The method according to claim 25, wherein activity is determined with a nitrocefin assay.

27. The method according to claim 22, wherein binding is measured with FACS.

28. A method of determining affinity, comprising:
i) immunizing a camelid;
ii) isolating at least one V_{H}H gene from the immunized camelid;
iii) fusing the at least one V_{H}H gene to a reporter gene, thereby creating at least one fusion gene;
iv) transforming the at least one fusion gene into a species that permits secretion of at least one fusion protein from the at least one fusion gene;
v) incubating the at least one fusion protein with at least one target; measuring affinity between the at least one target and the at least one fusion protein.
